(19) United States
(12) Plant Patent Application Publication
(10) Pub. No.: US 2003/0135906 P1
(43) Pub. Date: Jul. 17, 2003

(54) Petunia plant named 'SUNCHIFFON'

(76) Inventors: Kiyoshi Miyazaki, Hikone-shi (JP); Kazunari Iwaki, Ohmihachiman-shi (JP); Takuro Ishihara, Youkaichi-shi (JP)

Correspondence Address: BURNS, DOANE, SWECKER & MATHIS, L.L.P., P.O. Box 1404, Alexandria, VA, 22313-1404, US

(21) Appl. No.: 10/327,033

(22) Filed: Dec. 24, 2002

(30) Foreign Application Priority Data
Dec. 28, 2001 (JP) 14307

(51) Int. Cl. A01H 5/00

(52) U.S. Cl. PLT/356

(57) ABSTRACT

Disclosed herein is a new and distinct variety of Petunia plant having a decumbent habit and long stems. The Petunia plant has abundant branching, and great profusion of blooms, a whole plant remaining in bloom for a considerable period of time. The flowers are single and small in size, petals having a purplish pink color. The bottom color of the corolla throat is light greenish yellow and the outside of the corolla tube is pale greenish yellow. The plant exhibits high resistance to rain, heat, cold and disease.

### BACKGROUND OF THE VARIETY

The present invention relates to a new and distinct variety of petunia plant originated from crossing a seedling called ‘Fantasy Pink Morn’ (Goldsmith seeds Inc.) (♀) and a wild type Petunia plant called 40-1 (♂).

The Petunia is a very popular plant that is used for flower bedding and potting in the summer season. There are only a few Petunia varieties which do not have an upright growth habit and which have a high resistance to rain, heat, and diseases. The Petunia plant that we previously filed, i.e. the ‘Revolution’ series (‘Revolution Purple pink’ (U.S. Plant Pat. No. 6,915), ‘Revolution Brilliant pink’ (U.S. Plant Pat. No. 6,914), ‘Revolution Brilliant pink-mini’ (U.S. Plant Pat. No. 6,899), and ‘Revolution Blue vein’ (U.S. Plant Pat. No. 9,322)) are decumbent type plants having long stems, a lower plant height, abundant branching, and a high resistance to heat, rain and disease. However, there are only a few Petunia varieties having a great profusion of flowers, airily colored flower petals and a high resistance to rain, heat, and diseases. Accordingly, this invention is aimed at obtaining a new variety having purplish pink colored petals together with the above features.

The new variety of petunia plant according to this invention originated from crossing a seedling called ‘Fantasy Red Morn’ (♀) and a wild type Petunia plant called 40-1(♂). The seed of ‘Fantasy Pink Morn’ (not-patented in the United States) was bought from Sakata Seed Ltd. in Japan. 40-1 (not-patented in the United States) is a decumbent, wild type Petunia plant native to Brazil.

In April 1998, crossing of ‘Fantasy Red Morn’ as female parent and 40-1 as pollen parent was conducted at Omi R&D Center, Flower Division, Suntory Ltd. In January 1999, and 120 seedlings were obtained from that crossing. These seedlings were grown in pots in glasshouses and tested. One new variety was selected in view of its decumbent growth habit and its flower color. That seedling was propagated by cutting from July 1999, and a trial was carried out by flower potting and bedding from March 2000, at the Omi R&D Center,Flower Division, Suntory Ltd. The botanical characteristics of that plant were then examined, using similar varieties ‘Revolution pink-mini’ and ‘Sunpapi’ (not-patented in the United States) for comparison. As a result, it was concluded that this petunia plant is distinguishable from any other variety, whose existence is known to us, and is uniform and stable in its characteristics. Then the new variety of petunia plant was named ‘SUNCHIFFON’.

In the following description, the color-coding is in accordance with the Horticultural Color Chart of The Royal Horticultural Society, London, England (R.H.S. Color Chart). A color chart based on The Japan Color Standard for Horticultural Plants (J.H.S. Color Chart) is also added for reference.

The female parent used in the crossing of ‘SUNCHIFFON’, called ‘Fantasy Red Morn’, has an erect habit while that of the new variety is decumbent. The branching is fewer than the new variety. It has small single flowers, the petals having deep purplish pink (R.H.S. 68B, J.H.S. 9205) with pale purplish pink (R.H.S. 69D, J.H.S. 9501) morn. The bottom color of the corolla throat is yellowish white (R.H.S. 159D, J.H.S.1901) and the outside color of the corolla tube is yellowish white (R.H.S. 159D, J.H.S.1901).

The main botanical characteristics of the ‘Fantasy Red Morn’ are as follows:

Plant:

Growth habit.—Erect.

Plant height.—15.5 cm.

Spreading area of plant.—Small.

Blooming period.—April to July in the southern Kanto area, Japan.

Stem:

Length.—9.5 cm.

Thickness.—Main stem 2.8 mm.

Pubescence.—Sparse.

Branching.—Medium.

Length of internode.—2.2 cm.

Color.—Light yellow green (R.H.S. 145C, J.H.S. 3503).

Leaf:

Whole shape.—Elliptic. The apex shape is acute, and the base shape is attenuate.

Length.—3.2 cm.

Width.—2.2 cm.

Color.—Upper-side color is strong yellow green (R.H.S. 144A, J.H.S. 3507). Bottom-side color is moderate yellow green (R.H.S. 147B, J.H.S. 3513).

Pubescence.—Sparse.

Flower:

Facing direction.—Slightly slantly upward.

Type.—Single.

Shape.—Funnel-shape, with five-fissured limb.

Shape of petal chip.—Obtuse.

Lobation.—Medium.

Waving of petal.—Weak.

Diameter.—4.5 cm.

Color.—Petal; deep purplish pink (R.H.S. 68B, J.H.S.9205) with pale purplish pink (R.H.S. 69D, J.H.S.9501) morn. Bottom color of the corolla throat; yellowish white (R.H.S. 159D, J.H.S.1901). Outside color of the corolla tube; yellowish white (R.H.S. 159D, J.H.S.1901).

Reproductive organs.—1 normal pistil and 5 normal stamens. Color of pistil is vivid yellow green (R.H.S. 144B, J.H.S.3506). Color of stamen is pale yellow (R.H.S. 158C, J.H.S.2702).

Peduncle.—1.0 mm in thickness, and 1.0 cm in length.

Physiological and ecological characteristics.—Strong resistance to cold, rain, pests and disease.

The male parent 40-1 used in the crossing of ‘SUNCHIFFON’ was a wild type petunia plant native to Brazil. The petunia plant 40-1 has a decumbent habit with long stems and many branching. The leaf is very small without petiole. It has a small single flower, and pale purplish pink colored (R.H.S. 76D, J.H.S. 8902) petals. The bottom color of the corolla throat is yellowish white (R.H.S. 159D, J.H.S. 1901). The outside color of the corolla tube is yellowish white (R.H.S. 159D, J.H.S. 1901).

The main botanical characteristics of the 40-1 are as follows:

Plant:

Growth habit.—Decumbent.

Plant height.—5.5 cm.

Spreading area of plant.—Large.

Blooming period.—May to August in the southern Kanto area, Japan.

Stem:

Length.—16.0 cm.

Thickness.—1.5 mm.

Pubescence.—Normal.

Branching.—Many.

Length of internode.—1.5 cm.

Color.—Light yellow green (R.H.S. 145C, J.H.S. 3503).

Leaf:

Whole shape.—Elliptic. The apex shape is acute, and the base shape is attenuate.

Length.—2.7 cm.

Width.—1.7 cm.

Color.—Upper-side color is strong yellow green (R.H.S. 143A, J.H.S. 3711). Bottom-side color is moderate yellow green (R.H.S. 147B, J.H.S. 3513).

Pubescence.—Sparse.

Flower:

Facing direction.—Slantly upward.

Type.—Single.

Shape.—Funnel-shape, with five-fissured limb.

Shape of petal chip.—Round.

Lobation.—Shallow.

Waving of petal.—Weak.

Diameter.—4.1 cm.

Color.—Petal; pale purplish pink (R.H.S. 76D, J.H.S. 8902). Bottom color of the corolla throat; yellowish white (R.H.S. 159D, J.H.S.1901). Outside color of the corolla tube; yellowish white (R.H.S. 159D, J.H.S.1901).

Reproductive organs.—1 normal pistil and 5 normal stamens. Color of pistil is vivid yellow green (R.H.S. 144B, J.H.S.3506). Color of stamen is yellowish white (R.H.S. 155D, J.H.S.2501).

Peduncle.—1.1 mm in thickness, and 1.8 cm in length.

Physiological and ecological characteristics.—Moderate resistance to pests. Strong resistance to rain, cold, heat and diseases.

The main botanical characteristics of similar variety ‘Revolution pink-mini’ used for comparison are as follows:

Plant:

Growth habit.—Decumbent.

Plant height.—20 cm.

Spreading area of plant.—Large.

Blooming period.—May to August in the southern Kanto area, Japan.

Stem:

Length.—15 cm.

Thickness.—Main stem 2.5 mm.

Pubescence.—Normal.

Branching.—Many.

Length of internode.—2.0 cm.

Color.—Light yellow green (R.H.S. 145C, J.H.S. 3503).

Leaf:

Whole shape.—Elliptic. The apex shape is acute, and the base shape is attenuate.

Length.—5.0 cm.

Width.—3.0 cm.

Color.—Upper-side color is dark olive green (R.H.S.137A, J.H.S.3509). Bottom-side color is moderate yellow green (R.H.S. 147B, J.H.S. 3513).

Pubescence.—Normal.

Flower:

Facing direction.—Slantly upward.

Type.—Single.

Shape.—Funnel-shape, with five-fissured limb.

Shape of petal chip.—Round.

Lobation.—Shallow.

Waving of petal.—Weak.

Diameter.—4.5 cm.

Color.—Petal; Vivid reddish purple (R.H.S. 71D, J.H.S.9206). Bottom color of the corolla throat is pinkish white (R.H.S. 69D, J.H.S.9201). Outside color of the corolla tube is strong purplish pink (R.H.S. 73B, J.H.S.9204).

Reproductive organs.—1 normal pistil and 5 normal stamens. Color of pistil is vivid yellow green (R.H.S. 144B, J.H.S.3506). Color of stamen is yellowish white (R.H.S. 155D, J.H.S.2501).

Peduncle.—1.0 mm in thickness, and 2.0 cm in length.

Physiological and ecological characteristics.—Strong resistance to cold, heat, rain and diseases. Moderate resistance to pests.

The main botanical characteristics of similar variety ‘Sunpapi’ used for comparison are as follows:

Plant:

Growth habit.—Decumbent.

Plant height.—11.5 cm.

Spreading area of plant.—Large.

Blooming period.—April to September in the southern Kanto area, Japan.

Stem:

Length.—18.0 cm.

Thickness.—Main stem 3.2 mm.

Pubescence.—Normal.

Branching.—Many.

Length of internode.—2.2 cm.

Color.—Light yellow green (R.H.S. 145C, J.H.S. 3503).

Leaf:

Whole shape.—Lanceolate. The apex shape is acute, and the base shape is attenuate.

Length.—6.1 cm.

Width.—2.7 cm.

Color.—Upper-side color is dark olive green (R.H.S.137A, J.H.S. 3707). Bottom-side color is moderate yellow green (R.H.S. 147B, J.H.S. 3513).

Pubescence.—Normal.

Flower:

Facing direction.—Slantly upward.

Type.—Single.

Shape.—Funnel-shape, with five-fissured limb.

Shape of petal chip.—Obtuse.

Lobation.—Shallow.

Waving of petal.—Weak.

Diameter.—6.0 cm.

Color.—Petal; deep purplish pink (R.H.S. 68B, J.H.S. 9205). Bottom color of the corolla throat; yellowish white (R.H.S. 159D, J.H.S.1901). Outside color of the corolla tube; yellowish white (R.H.S. 159D, J.H.S.1901).

Reproductive organs.—1 normal pistil and 5 normal stamens. Color of pistil is vivid yellow green (R.H.S. 144B, J.H.S.3506). Color of stamen is yellowish white (R.H.S. 155D, J.H.S.2501).

Peduncle.—1.4 mm in thickness, and 1.8 cm in length.

Physiological and ecological characteristics.—Strong resistance to cold, heat, rain and diseases. Moderate resistance to pests.

### SUMMARY OF THE VARIETY

The new variety of the petunia plant has a decumbent growth habit, abundant branching and great profusion of blooms with the whole plant remaining in bloom for a considerable period of time. The flowers are single and small in size. The petal color is purplish pink. The plant has a high resistance to rain, cold, heat and diseases.

### BRIEF DESCRIPTION OF THE PHOTOGRAPHS

FIG. 1 is a photograph of the new variety of Petunia plant, ‘SUNCHIFFON’, planted in a flower pot.

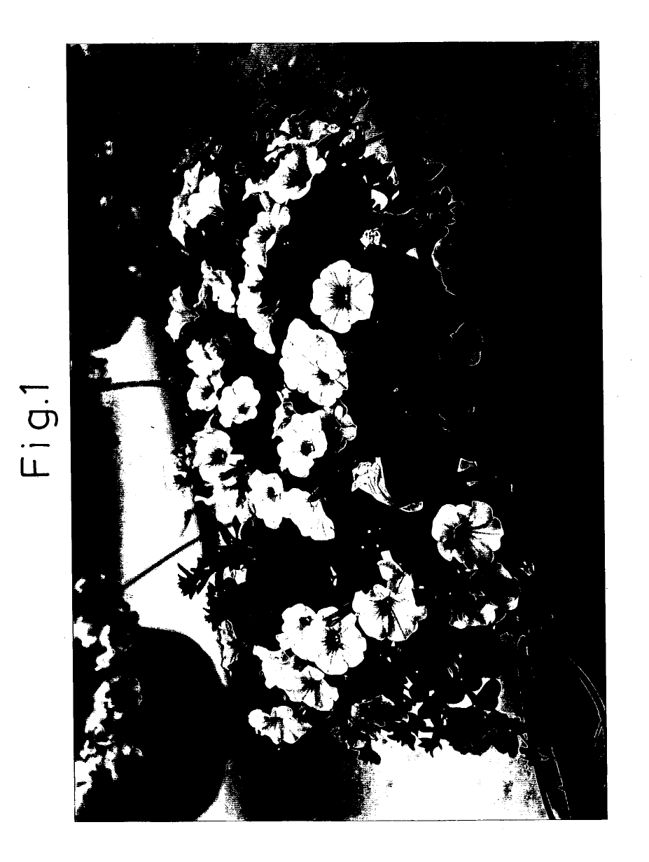

FIG. 2 is a photograph of flowers of the new variety of Petunia plant, ‘SUNCHIFFON’.

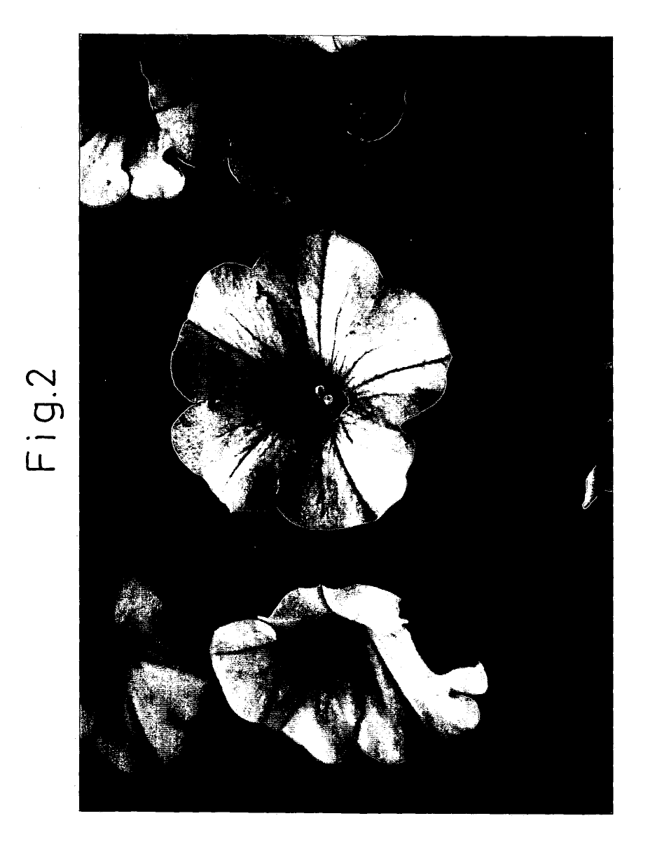

### DESCRIPTION OF THE NEW VARIETY

The botanical characteristics of the new and distinct variety of Petunia plant named ‘SUNCHIFFON’ are as follows.

Plant:

Growth habit.—Decumbent.

Plant height.—18 cm.

Spreading area of plant.—Large.

Blooming period.—Early April to late October in the southern Kanto area, Japan. The plant shape does not change throughout this period. A typical flower commonly lasts approximately 5 days on the plant when experiencing a temperature of approximately 20° C.

Stem:

Length.—15 cm.

Thickness.—2.0 mm.

Pubescence.—Normal.

Branching.—Many.

Length of internode.—2.0 cm.

Color.—Light yellow green (R.H.S. 145C, J.H.S. 3503).

Leaf:

Whole shape.—Slightly ovate. The apex shape is acute, and the base shape is attenuate.

Length.—4.0 cm.

Width.—2.8 cm.

Color.—Upper side color is moderate olive green (R.H.S. 146A, J.H.S. 3508). Bottom side color is moderate yellow green (R.H.S. 147B, J.H.S. 3513).

Thickness.—0.8 mm.

Pubescence.—Sparse.

Flower:

Facing direction.—Upward.

Type.—Single.

Shape.—Funnel-shape, with five-fissured limb.

Shape of petal chip.—Round.

Lobation.—Shallow.

Waving of petal.—Weak.

Diameter.—4.5 cm.

Color.—Petal; moderate purplish pink (R.H.S. 68C, J.H.S. 9211). Bottom color of the corolla throat; light greenish yellow (R.H.S. 1D, J.H.S. 2904). Outside color of the corolla tube; pale greenish yellow (R.H.S. 8D, J.H.S. 2903).

Reproductive organs.—1 normal pistil and 5 normal stamens. Color of pistil is vivid yellow green (R.H.S. 144B, J.H.S.3506). Color of stamen is yellowish white (R.H.S. 155D, J.H.S.2501).

Peduncle.—1.2 mm in diameter and 1.5 cm in length.

Physiological and ecological characteristics.—High resistance to rain, cold, heat and diseases. Moderate resistance to pests.

This new variety of Petunia plant is most suitable for flower bedding and potting, particularly in hanging pots or planters, and is excellent for use as ground cover. Pinching of old blossoms will enhance the formation of new blossoms.

I claim:

1. A new and distinct variety of Petunia plant, substantially as herein illustrated and described, characterized particularly as to novelty by (A) having a decumbent habit with long stems, (B) an abundant branching and great profusion of blooms, a whole bush remaining in bloom for a considerable period of time, (C) flowers are single and small in size, petals have a purplish pink color, and the bottom color of the corolla throat is light greenish yellow and the outside color of corolla tube is pale greenish yellow, and (D) a high resistance to rain, heat, cold and diseases.
